(19) United States
(12) Patent Application Publication
(10) Pub. No.: US 2021/0113723 A1
(43) Pub. Date: Apr. 22, 2021

(54) UV-SANITIZING APPARATUSES AND SYSTEMS

(71) Applicant: Raffel Systems, LLC, Germantown, WI (US)

(72) Inventors: Jeff Malinowski, Slinger, WI (US); Morgan Balog, Milwaukee, WI (US); Justin R. Saul, Milwaukee, WI (US)

(21) Appl. No.: 16/871,960

(22) Filed: May 11, 2020

Related U.S. Application Data
(60) Provisional application No. 62/916,428, filed on Oct. 17, 2019.
(60) Provisional application No. 63/001,042, filed on Mar. 27, 2020.

(51) Int. Cl. A61L 2/10; A47G 23/02

(52) U.S. Cl. CPC A61L 2/10; A47G 23/0216; A61L 2202/14; A61L 2202/122; A61L 2202/11

(57) ABSTRACT

The present invention relates generally to ultraviolet (UV)-sanitizing apparatuses and systems. More specifically, the invention relates to multifunctional UV-sanitizing apparatuses and systems containing a germicidal light source for use in private or public locations for use in, for examples, killing or inactivating pathogenic microorganisms and/or preventing the spread of disease.

### CROSS REFERENCE TO RELATED APPLICATION

This application claims priority to and the benefit of U.S. Provisional Application No. 62/916,428, filed Oct. 17, 2019, and U.S. Provisional Application No. 63/001,042, filed Mar. 27, 2020, the contents of which are hereby incorporated by reference in their entireties.

### FIELD

The present invention relates generally to ultraviolet (UV)-sanitizing apparatuses and systems. More specifically, the invention relates to multifunctional, UV-sanitizing apparatuses and systems containing a germicidal light source for use in private or public locations for use in, for examples, killing or inactivating pathogenic microorganisms and/or preventing the spread of disease.

### BACKGROUND

Many objects are placed onto surfaces in private and public environments, including water bottles, cell phones, pencils, remote controls, trash, and the like. These objects, particularly cell phones and remote controls, may harbor or serve as a reservoir for an abundance of harmful, pathogenic microorganisms including bacteria and viruses. As such, subsequent users may inadvertently come into contact with pathogenic microorganisms when using these objects or when contacting surfaces that have come into contact with these objects thereby leading to the spread of illnesses caused by the microorganisms. This problem is particularly problematic in public areas, such as airports, airplanes and other means of public transportation, healthcare settings, hotels, dormitories and other temporary living environments, restaurants, movie theaters, and the like.

### SUMMARY

Provided herein are UV-sanitizing apparatuses and systems containing a germicidal light source for use in private or public locations for use in, for examples, killing or inactivating pathogenic microorganisms and/or preventing the spread of disease. In particular, disclosed herein are multifunctional apparatuses and systems containing at least one surface, one or more means for charging a device, and one or more germicidal light sources. The one or more germicidal light sources may be disposed on the at least one surface for sanitizing an apparatus or object placed within an apparatus (e.g., described herein). Where an apparatus comprises a compartment or chamber (e.g., a sleeve, pocket, shelf, or box), the one or more germicidal light sources may be disposed on a surface within the compartment or chamber. In some embodiments, the apparatus contains two or more germicidal light sources. The invention is not limited to any particular germicidal light source. Indeed, any germicidal light source may be used including but not limited to an ultraviolet (UV) light source. In some embodiments, the germicidal light source produces 0.001 to 20 Watts of UV-C light (e.g., 0.001, 0.01, 0.1, 1, 2, 5, 10, 15, 20 Watts, or any wattage therebetween). The disclosure is not so limited, however. Any wattage of UV light may be used including but not limited to a wattage below 0.001 Watts, a wattage between 0.001-1.0 Watts, a wattage between 1-10 Watts or 1-20 Watts, or a wattage above 20 Watts.

The disclosure is not limited to the type of UV light source used to disinfect. Short wavelength ultraviolet light may be used (e.g., light with wavelengths between about 200 nm and 300 nm may be used, or light with shorter (e.g., less than 200 nm) or longer (e.g., greater than 300 nm) wavelengths may be used). Any light with germicidal properties may be used. Germicidal light sources may be controlled manually (e.g., via a switch on or within the multifunctional apparatus) or autonomously (e.g., using a system described herein). Similarly, where a plurality of germicidal light sources are present on or within an apparatus disclosed herein, or where a plurality of apparatuses are interconnected to be in communication (e.g., wired or wireless communication) with each other or with a system containing the apparatuses, the plurality of germicidal light sources may be controlled manually or autonomously. The plurality of germicidal light sources may be controlled simultaneously or individually. In some embodiments, the disclosure provides systems harboring a plurality of apparatuses or a plurality of objects (e.g., desks, nightstands, beds, etc.) to which the apparatuses are fastened, further comprising a UV light monitoring, recording and notification system comprising means for detecting, recording and optionally notifying that disinfecting UV light has been applied (e.g., to an apparatus, plurality of apparatuses, or to an object to which the apparatus is fastened. While the disclosure is not limited to any particular monitoring, recording and notification system, in some embodiments, the system comprises a controller that monitors and records UV light source use information (e.g., of UV sanitizing light of a multifunctional UV-sanitizing apparatus and/or UV sanitizing light separate from a UV sanitizing apparatus). In some embodiments, the controller activates an alert (e.g., visual (e.g., illumination of a light emitting diode) and/or audio (e.g., tone) alert) that indicates that UV sanitizing light has been activated and exposed for a period of time (e.g., to an apparatus, device or object placed in or on a multifunctional UV sanitizing apparatus).

The one or more means for charging a device of a multifunctional apparatus disclosed herein may be any means for charging a device including, but not limited to, wired and wireless charging means. Examples of charging using a wired connection include but are not limited to cables, cords, and the like. In some embodiments, a multifunctional apparatus contains one or more USB charging receptacles (e.g., whereby a device is charged using a USB or micro USB cord and USB receptacle). Wireless charging means include any type of charging via electromagnetic induction. Wireless charging means may use any standard in the wireless charging industry designed for portable devices including, but not limited to, Wireless Power Consortium's Qi and the PMA or AirFuel Alliance standards.

A means for charging a device of a multifunctional apparatus disclosed herein can be used to charge any type of device including, but not limited to, a mobile phone (e.g., smartphone), tablet, iPad, computer, or any other device chargeable via wired or wireless means.

A multifunctional apparatus disclosed herein may further comprise one or more controls for controlling one or more components separate from multifunctional apparatus. For example, a multifunctional cupholder apparatus integrated into a piece of powered furniture (e.g., a seating arrangement such as a powered sofa, loveseat, or chair) may comprise one or more controls (e.g., switches) that control a component of the piece of furniture including, but not limited to, a powered recline mechanism, a massage mechanism, a powered headrest mechanism, and/or lighting components of the furniture. In like manner, a multifunctional shelf apparatus attached to or integrated into a piece of furniture may contain one or more controls for controlling one or more components separate from the shelf apparatus including, but not limited to, a powered recline mechanism, a massage mechanism, a powered headrest mechanism, and/or lighting components of the furniture.

A multifunctional pocket apparatus attached to or integrated into a case good such as a nightstand or desk may contain a control for controlling the germicidal light sources on or within the pocket and one or more controls for controlling one or more components separate from the pocket apparatus including but not limited to night, reading, or ambient lighting. Case goods include but are not limited to nightstands, desks, dressers, bedframes, box springs, head boards, shelving units, walls, counters, tv stands/entertainment centers, chairs (e.g., non-upholstered chair), airplane seats, train seats, bus seats, subway seats, and structures/apparatuses found in recreational vehicles (RVs) or boats. In like manner, a case good may have one or more control switches. In some embodiments, the one or more control switches of the multifunctional apparatus and the one or more control switches of the case good control the same or different component. In further embodiments, the one or more control switches of the multifunctional apparatus and the one or more control switches of the case good are in operable communication with each other (e.g., via a controller).

In some embodiments, a multifunctional apparatus further contains one or more sensors. For example, the apparatus may further contain an infrared sensor, a proximity sensor, a pressure sensor, or a combination thereof. The one or more sensors may be disposed on the at least one surface of the apparatus.

In some embodiments, a multifunctional apparatus further contains a timer.

Systems are provided containing one of more multifunctional apparatuses (e.g., comprising at least one surface, one or more means for charging a device, and one or more germicidal light sources), and a controller (e.g., comprising a processor and a memory component). In some embodiments, the controller operates to control the one or more germicidal light sources (e.g., via running software stored in the memory component or elsewhere (e.g., cloud-based storage)). The controller can be configured to control a plurality of apparatuses and/or a plurality of germicidal light sources present on or within the apparatuses simultaneously or individually. For example, in an environment containing a plurality of rooms each containing a piece of furniture (e.g., a hotel or dormitory with each room containing a desk, nightstand and/or upholstered furniture), wherein the piece of furniture contains one or more multifunctional apparatuses (e.g., comprising at least one surface, one or more means for charging a device, and one or more germicidal light sources), a controller of a system described herein can be configured to control the plurality of germicidal light sources present on or within the apparatuses simultaneously or individually. In some embodiments, in addition to controlling germicidal light sources present on or within the apparatuses, the controller may be configured to control one or more separate germicidal light sources not on or within an apparatus disclosed herein (e.g., in a hotel or dormitory setting, a germicidal light source located on a ceiling, a floor, a wall, a counter, or on a desk may be operably connected (e.g., wired or wirelessly connected) to a controller). In some embodiments, a controller further comprises a communication means (e.g., to send or receive information wirelessly). In further embodiments, the controller receives input from the one or more germicidal light sources, the one or more sensors, and/or one or more timers (e.g., to activate or deactivate the one or more germicidal light sources). In still further embodiments, the controller sends information to the one or more germicidal light sources, the one or more sensors, and/or one or more timers (e.g., if the controller processes received information that the germicidal light has been activated for a period of time, the controller then sends a signal to the light source to activate an alert (e.g., a green indicator light) on the light source to provide an alert to an individual that the surfaces exposed to the light have been decontaminated).

### BRIEF DESCRIPTION OF THE FIGURES

The following drawings form part of the present specification and are included to further illustrate aspects of the disclosure. The drawings highlight exemplary embodiments of the disclosure, but should not be viewed as limiting the scope thereof. The disclosure may be better understood by reference to the drawings in combination with the detailed description of the specific embodiments presented herein.

FIG. 1 shows a top view image of an exemplary multifunctional cupholder apparatus containing one or more germicidal light sources.

FIG. 2 shows a side view image of an exemplary multifunctional cupholder apparatus containing an on/off switch that controls the one or more germicidal light sources.

FIG. 3 is an image showing bacterial-killing capability of the multifunctional, UV-sanitizing cupholder apparatuses described herein. Cupholders were swabbed before and after UV-sanitization and bacteria were grown on LB agar plates. The image on the left shows the number of bacterial colonies before UV sanitization. The image on the right shows the number of bacterial colonies after UV sanitization. Following sanitization, 99% of the bacteria were killed.

FIG. 4 depicts an exemplary multifunctional cupholder apparatus containing a cupholder, wireless charging sleeve, germicidal light sources, and a control for controlling the germicidal light sources.

FIG. 5 depicts an exemplary multifunctional shelf apparatus (e.g., for attachment to or integration into a piece of furniture) containing a chamber, germicidal light sources, a plurality of wired charging means, wireless charging means, a control for controlling the germicidal light sources, and a plurality of controls for controlling one or more components separate from the shelf apparatus (e.g., a recline mechanism of a piece of powered upholstered furniture).

FIG. 6 depicts an exemplary multifunctional clamshell apparatus (e.g., for attachment to a topside or underside of a surface (e.g., a desk, a furniture console, a wall) containing a compartment, germicidal lights, wired charging means, and wireless charging means.

FIG. 7 depicts an exemplary multifunctional pocket apparatus (e.g., for attachment to or integration into a case good such as a nightstand or desk) containing a chamber, germicidal light sources, a plurality of wired charging means, wireless charging means, a control for controlling the germicidal light sources, and a plurality of controls for controlling one or more components separate from the pocket apparatus (e.g., night, reading, or ambient lighting).

FIG. 8 depicts an exemplary multifunctional sleeve apparatus (e.g., for use in public transportation such as airplanes) containing a compartment, germicidal light sources, a plurality of wired charging means, wireless charging means, a control for controlling the germicidal light sources. The blowout (right) shows the sleeve compartments at it exists internally when the apparatus is in a closed position (left).

FIG. 9 depicts an exemplary multifunctional sleeve apparatus containing a chamber, germicidal light sources, a wired charging means, surface lighting, and a plurality of controls for controlling one or more components separate from the pocket apparatus (e.g., night, reading, ambient lighting, or mechanism of a piece of powered upholstered furniture).

### DEFINITIONS

As used herein, the term “germicidal” refers to the capacity to kill microbial organisms such as bacteria, viruses, parasites, fungi, yeast, and molds.

As used herein, the term “pathogen” refers to an organism that can cause disease. For example, the term “pathogens” may refer to bacteria, viruses, parasites, fungi, yeast, molds, or other infectious organisms.

As used herein, the term “UV” or “ultraviolet” refers to light with a wavelength following between about 100 and about 400 nanometers.

As used herein, the term “UV-A” or “UV-A light” refers to UV light of about 320-400 nm in wavelength.

As used herein, the term “UV-B” or “UV-B light” refers to UV light of about 290-320 nm in wavelength.

As used herein, the term “UV-C” or “UV-C light” refers to UV light having a wavelength of about 100 nanometers (nm) to about 290 nm.

As used herein, the term “user” or “operator” refers to an individual who utilizes a UV-sanitizing apparatus as described herein.

As used herein the term “processor” refers to a device that is able to read a program from a computer memory (e.g., ROM or other computer memory) and perform a set of steps according to the program. Processor may include non-algorithmic signal processing components (e.g., for analog signal processing). In some embodiments, a controller of the disclosures comprises a processor (e.g., a microprocessor) and a memory component (e.g., a flash memory component). Examples of controllers useful in the disclosure include, but are not limited to, a 32 bit microcontroller (e.g., that utilizes a reduced instruction set computing (RISC) microprocessor). In some embodiments, a controller integrates a 12-bit analog-to-digital converter (ADC), queued serial peripheral interfaces (QSPI), and/or a four channel general purpose timer (GPT) (e.g. capable of pulse width modulation (PWM)). Any controller comprising one or more of the functions described may be utilized herein.

As used herein, the terms “memory component,” “computer memory” and “computer memory device” refer to any storage media readable by a computer processor. Examples of computer memory include, but are not limited to, RAM, ROM, computer chips, digital video disc (DVDs), compact discs (CDs), hard disk drives (HDD), and magnetic tape, any of the foregoing being local (e.g., at the same location as the processor or multifunctional apparatus) or remote (e.g., at a different location as the processor or multifunctional apparatus (e.g., in the cloud)).

As used herein, the term “computer readable medium” refers to any device or system for storing and providing information (e.g., data and instructions) to a computer processor. Examples of computer readable media include, but are not limited to, DVDs, CDs, hard disk drives, magnetic tape, flash memory, and servers for streaming media over networks.

As used herein the terms “multimedia information” and “media information” are used interchangeably to refer to information (e.g., digitized and analog information) encoding or representing audio, video, and/or text. Multimedia information may further carry information not corresponding to audio or video. Multimedia information may be transmitted from one location or device to a second location or device by methods including, but not limited to, electrical, optical, and satellite transmission, and the like.

As used herein, the term “Internet” refers to any collection of networks using standard protocols. For example, the term includes a collection of interconnected (public and/or private) networks that are linked together by a set of standard protocols (such as TCP/IP, HTTP, and FTP) to form a global, distributed network. While this term is intended to refer to what is now commonly known as the Internet, it is also intended to encompass variations that may be made in the future, including changes and additions to existing standard protocols or integration with other media (e.g., television, radio, etc.). The term is also intended to encompass non-public networks such as private (e.g., corporate) Intranets.

As used herein the term “security protocol” refers to an electronic security system (e.g., hardware and/or software) to limit access to processor, memory, etc. to specific users authorized to access the processor. For example, a security protocol may comprise a software program that locks out one or more functions of a processor until a certain event occurs (e.g., until an appropriate password is entered, authorized radio-frequency identification (RFID) tag is presented, proper biometric match is made, or the like).

As used herein the term “resource manager” refers to a system that optimizes the performance of a processor or another system. For example, a resource manager may be configured to monitor the performance of a processor or software application and manage data and processor allocation, perform component failure recoveries, optimize the receipt and transmission of data, and the like. In some embodiments, the resource manager comprises a software program provided on a computer system of the present disclosure.

As used herein the terms “in electronic communication” and “in communication with” refers to electrical devices (e.g., computers, processors, communications equipment) that are configured to communicate with one another through direct or indirect signaling. For example, a conference bridge that is connected to a processor through a cable or wire, such that information can pass between the conference bridge and the processor, are in electronic communication with one another. Likewise, a computer configured to transmit (e.g., through cables, wires, infrared signals, telephone lines, etc.) information to another computer or device, is in electronic communication with the other computer or device.

As used herein the term “transmitting” refers to the movement of information (e.g., data) from one location to another (e.g., from one device to another) using any suitable means.

As used herein, the terms “hotel,” “motel,” “lodge,” “hostel,” “dormitory,” and the like are used interchangeably to refer to a building in which individuals stay or reside on a temporary basis.

### DETAILED DESCRIPTION

The disclosure provides multifunctional, UV sanitizing apparatuses and systems. Multifunctional apparatuses contain a germicidal light source that may be used to disinfect the apparatus and/or objects placed within the apparatus. In this way, the multifunctional, UV-sanitizing apparatuses described herein reduce and/or eliminate the microbial (e.g., pathogenic microbe) load on the apparatus and/or objects placed within the same, thus preventing the spread of illness. Furthermore, the multifunctional UV-sanitizing apparatuses described herein are environmentally friendly options for sanitization. For example, the multifunctional UV-sanitizing apparatuses described herein eliminate the need for traditional cleaning agents (e.g. soaps, sanitizing liquids, sanitizing wipes, sponges, paper towels, etc.), and the containers used to house and ship same, thus reducing waste from such agents.

In some embodiments, the multifunctional UV-sanitizing apparatus comprises at least one surface. The apparatus may comprise any one or more suitable surfaces that come into contact with or that are placed next to or within close proximity to a device or object harboring microbes (e.g., microbial pathogens). For example, the multifunctional apparatus may comprise one or more surfaces that comes into contact with or that are placed next to or within close proximity to a device or an object harboring the microbes (e.g., microbial pathogens). Exemplary devices or objects include, for example, cell phones, tablets, laptop computers, remote controls, keys, wallets, cords, food or beverage containers or products, books, magazines, trays, tables (e.g., a tray or table used on aircraft seating), newspapers, writing instruments, and the like.

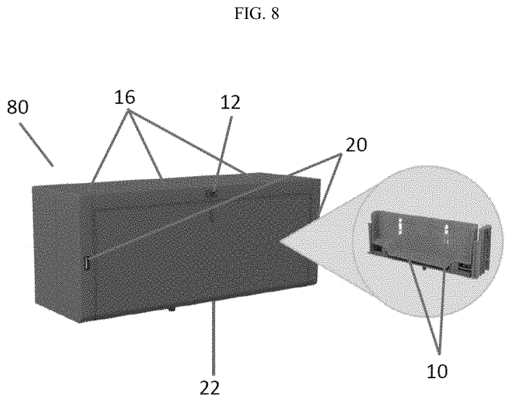

In some embodiments, the multifunctional apparatus comprises a single flat surface (e.g. a tray). In some embodiments, the apparatus comprises a bottom surface and at least one side surface. For example, the apparatus may comprise a circular bottom surface and one rounded side surface, such as a cupholder. In some embodiments, the apparatus comprises a bottom surface and at least two side surfaces (e.g. 2 side surfaces, 3 side surfaces, 4 side surfaces, etc.). In some embodiments, the apparatus comprises a bottom surface that is square or rectangular in shape, and four side surfaces that intersect to form a container such as a shelf, a sleeve, or a pocket. In some embodiments, the apparatus is an open container comprising a bottom surface, at least one side surface, and no top surface. For example, the apparatus may be a container such as a cupholder, a sleeve, a shelf, or a pocket. In some embodiments, the apparatus is a closable container comprising a bottom surface, at least one side surface, and a lid. The lid may be retractable or removable to allow for placement of an object within the apparatus and subsequent closure of the apparatus. When in a closed position, the inner surface of the lid provides a top surface for the apparatus itself. For example, the apparatus may be a closable container such as a box or a clamshell, containing a bottom surface, four side surfaces, and a lid that provides a top surface when in the closed position. Exemplary multifunctional UV-sanitizing apparatuses are provided in the accompanying examples and drawings. Note that the term “top surface” as used herein does not refer to the orientation of the apparatus itself, but rather refers solely to the surface resulting from closure of a lid on the apparatus. For example, as shown in FIG. 8 the apparatus may comprise a retractable lid. Although the retractable lid is shown on the side of the apparatus, it is understood that the inner surface generated as a result of closing the lid is referred to herein as a “top surface”.

The multifunctional UV-sanitizing apparatus comprises at least one germicidal light source. The germicidal light source may be a UV light source. UV light is divided into UV-A, UV-B, and UV-C light based upon wavelength. UV-C light is a germicidal ultraviolet light with proven effectiveness in inactivating or killing a very wide range of viruses, bacteria, protozoa, yeast, mold, and other microbial pathogens. Specifically, the mechanism by which germicidal light kills microorganisms is by damaging the deoxyribonucleic acid (DNA), of the microorganism. In the inactivation process, the thymine base of the DNA contains an aromatic ring that strongly absorbs the wavelength of the ultraviolet light, such as the UV-C light. When enough ultraviolet light has been absorbed, the chemical bond is broken between the thymine base and its base pair across the DNA strand. The thymine base then chemically bonds to a neighboring base, creating a thymine dimer, which prevents the replication of the microorganisms (e.g., pathogenic microbes). In particular embodiments, the at least one germicidal light source is a source of UV-C light (e.g., short UV-C light).

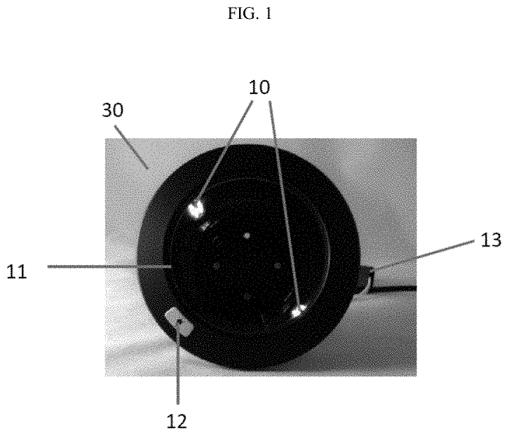

The germicidal light source may be placed in any suitable location on or within the multifunctional apparatus. The exact location on or within the apparatus will depend on the specific type of apparatus. For example, the germicidal light source may be disposed on the single flat surface (e.g., on the surface of a tray, table, or otherwise flat surface) of the apparatus. In some embodiments, the one or more germicidal light sources may be disposed within a container (e.g. a cupholder, a sleeve, a shelf, a pocket, a box, a clamshell, etc.). For example, the germicidal light source may be placed on an inner surface of a container, such that the germicidal light source exposes a majority or the entirety of the inside of the container to the germicidal light when activated. For example, the germicidal light source may be placed on an inner surface (e.g. a bottom surface, a side surface, or a top surface) of a cupholder, a sleeve, a shelf, a pocket, a box, a clamshell, etc. Exemplary locations of the germicidal light source are shown in the drawings. For example, as shown in FIG. 1 two germicidal light sources are disposed within the cupholder (e.g. on an inner surface of the container) such that activation of the germicidal light sources enables exposure of the entire inside of the container (e.g. inner bottom surface and inner side surface) to the germicidal light. For open containers (e.g. cupholders, sleeves, shelves, pockets, etc.) the germicidal light source may be disposed on any suitable inner surface (e.g. bottom surface or side surface). For closable containers (e.g. boxes, clamshells, etc.) the germicidal light source may be disposed on the bottom surface, side surface, or on the inner surface of the lid (e.g. top surface), such that when the container is closed the insides of the container are exposed to the germicidal light. In instances where multiple germicidal light sources are used, the lights may be placed in any suitable combination of locations to maximize exposure of the apparatus to the germicidal light.

The germicidal light source may be any suitable light source. Exemplary germicidal light sources include, for example, light bulbs (e.g. LEDs), light strips, lamps, etc. In some embodiments of the present disclosure, a germicidal UV light source is a low-pressure UV lamp. Low-pressure UV lamps are similar to a fluorescent lamp, with a wavelength of 253.7 nm. Low pressure lamps are effective because they emit most of the radiant energy in the germicidal wavelength of 253.7 nm (e.g. the UV-C part of the spectrum). Any type of UV lamps including low pressure, medium pressure, high pressure, and ultra-high-pressure lamps can be used. Additionally, spectral calibration lamps, electrodeless lamps, and the like can be used. In some embodiments, the germicidal UV light source is a UV-C LED bulb. In other embodiments, a plurality of light sources emitting the same or different wavelengths of light are used.

The multifunctional UV-sanitizing apparatus may include one germicidal light source or multiple germicidal light sources. For example, the UV-sanitizing apparatus may include two, three, four, five, six, seven, eight, nine, ten, or more than 10 germicidal light sources. In some embodiments, the UV-sanitizing apparatus may include a combination of different germicidal light sources (e.g. low-pressure UV lamps and one or more different sources). As described herein, one or more UV-sanitizing apparatuses containing one or more germicidal light sources may be operably connected to one or more other germicidal light sources (e.g., germicidal light source or sources located on a ceiling, a wall, a shelf, a counter, an office cubical or partition, a desk, or a floor) separate from those of the UV-sanitizing apparatuses.

The germicidal light source may produce any suitable amount of germicidal (e.g. UV-C) light for any suitable duration to kill/inactivate the desired microbes (e.g., pathogens). For example, the germicidal light source may be used appropriately to kill or inactivate microbes (e.g., pathogenic microbes) including bacteria (e.g. gram positive bacteria, gram negative bacteria), viruses (e.g. enveloped viruses, non-enveloped viruses), parasites, fungi, yeast, molds, or other infectious organisms. In some embodiments, the light source or sources are controlled individually or in combination (e.g., using a timer or a controller).

Exemplary bacteria that may be killed or inactivated by exposure to the germicidal light include, for example, Acetobacter aurantius, Acinetobacter baumannii, Actinomyces israelii, Agrobacterium (e.g. Agrobacterium radiobacter, Agrobacterium tumefaciens), Anaplasma phagocytophilum, Azorhizobium caulinodans, Azotobacter vinelandii, viridans streptococci, Bacillus (e.g. Bacillus anthracis, Bacillus brevis, Bacillus cereus, Bacillus fusiformis, Bacillus licheniformis, Bacillus megaterium, Bacillus mycoides, Bacillus stearothermophilus, Bacillus subtilis, Bacillus thuringiensis), Bacteroides (e.g. Bacteroides fragilis, Bacteroides gingivalis, Bacteroides melaninogenicus), Bartonella (e.g. Bartonella henselae, Bartonella Quintana), Bordetella (e.g. Bordetella bronchiseptica, Bordetella pertussis, Borrelia burgdorferi), Brucella (e.g. Brucella abortus, Brucella melitensis, Brucella suis), Burkholderia (e.g. Burkholderia mallei, Burkholderia pseudomallei, Burkholderia cepacian), Calymmatobacterium granulomatis, Campylobacter (e.g. Campylobacter coli, Campylobacter fetus, Campylobacter jejuni, Campylobacter pylori), Chlamydia trachomatis, Chlamydophila (e.g. Chlamydophila pneumoniae, Chlamydophila psittaci), Clostridium (e.g. Clostridium botulinum, Clostridium difficile, Clostridium perfringens, Clostridium tetani), Corynebacterium (e.g. Corynebacterium diphtheriae, Corynebacterium fusiforme), Coxiella burnetiid, Ehrlichia chaffeensis, Ehrlichia ewingii, Eikenella corrodens, Enterobacter cloacae, Enterococcus (e.g. Enterococcus avium, Enterococcus durans, Enterococcus faecalis, Enterococcus faecium, Enterococcus gallinarum, Enterococcus maloratus), Escherichia coli, Fusobacterium necrophorum, Fusobacterium nucleatum, Gardnerella vaginalis, Haemophilus (e.g. Haemophilus ducreyi, Haemophilus influenzae, Haemophilus parainfluenzae, Haemophilus pertussis, Haemophilus vaginalis), Helicobacter pylori, Klebsiella pneumoniae, Lactobacillus (e.g. Lactobacillus acidophilus, Lactobacillus bulgaricus, Lactobacillus casei), Lactococcus lactis, Legionella pneumophila, Leishmania donovani, Leptospira interrogans, Leptospira noguchii, Listeria monocytogenes, Methanobacterium extroquens, Microbacterium multiforme, Micrococcus luteus, Moraxella catarrhalis, Mycobacterium (e.g. Mycobacterium avium, Mycobacterium bovis, Mycobacterium diphtheriae, Mycobacterium intracellulare, Mycobacterium leprae, Mycobacterium lepraemurium, Mycobacterium phlei, Mycobacterium smegmatis, Mycobacterium tuberculosis), Mycoplasma (e.g. Mycoplasma fermentans, Mycoplasma genitalium, Mycoplasma hominis, Mycoplasma penetrans, Mycoplasma pneumoniae, Mycoplasma Mexican), Neisseria (e.g. Neisseria gonorrhoeae, Neisseria meningitidis) Pasteurella (e.g. Pasteurella multocida, Pasteurella tularensis), Peptostreptococcusm, Porphyromonas gingivalis, Prevotella melaninogenica, Pseudomonas aeruginosa, Rhizobium radiobacter, Rickettsia (e.g. Rickettsia prowazekii, Rickettsia psittaci, Rickettsia quintana, Rickettsia, Rickettsia trachoma), Rochalimaea (e.g. Rochalimaea henselae, Rochalimaea quintana), Rothia dentocariosa, Salmonella (e.g. Salmonella enteritidis, Salmonella typhi, Salmonella typhimurium), Serratia marcescens, Shigella dysenteriae, Spirillum volutans, Staphylococcus (e.g. Staphylococcus aureus, Staphylococcus epidermidis), Stenotrophomonas maltophilia, Streptococcus (e.g. Streptococcus agalactiae, Streptococcus avium, Streptococcus bovis, Streptococcus cricetus, Streptococcus faceium, Streptococcus faecalis, Streptococcus ferus, Streptococcus gallinarum, Streptococcus lactis, Streptococcus mitior, Streptococcus mitis, Streptococcus mutans, Streptococcus oralis, Streptococcus pneumoniae, Streptococcus pyogenes, Streptococcus rattus, Streptococcus salivarius, Streptococcus sanguis, Streptococcus sobrinus), Treponema (e.g. Treponema pallidum, Treponema denticola), Thiobacillus, Ureaplasma urealyticum, Vibrio (e.g. Vibrio cholerae, Vibrio comma, Vibrio parahaemolyticus, Vibrio vulnificus), Wolbachia, Yersinia (e.g. Yersinia enterocolitica, Yersinia pestis, Yersinia pseudotuberculosis).

Exemplary viruses that may be killed or inactivated by the germicidal light source include, for example, adenoviridae (e.g. Adenovirus), arenaviridae (e.g. Lassa virus), astroviridae (e.g. Human astrovirus), bunyavirida (e.g. Crimean-Congo hemorrhagic fever virus, Hantaan virus), Caliciviridae (e.g. Norwalk virus), coronaviridae (e.g. coronavirus OC43, coronavirus 229E, coronavirus NL63, coronavirus HKU1, MERS-CoV, SARS-CoV, and SARS-CoV-2 (COVID-19)), filoviridae (e.g. Ebola virus, Marburg virus), flaviviridae (e.g. Hepatitis C virus, yellow fever virus, dengue virus, west nile virus, TBE virus), hepadnaviridae (e.g. Hepatitis B virus), hepeviridae (e.g. Hepatitis E virus), Herpesviridae (e.g. HSV1, HSV2, varicella-zoster virus, Eppstein-Barr virus, Human cytomegalovirus, Human herpesvirus), Orthomyxoviridae (e.g. influenza A virus, influenza B virus), papilloviridae (e.g. human papillomavirus), paramyxoviridae (e.g. measles virus, mumps virus, parainfluenza virus type 1, parainfluenza virus type 2, respiratory syncytial virus), parvoviridae (e.g. parvovirus), picornaviridae (e.g. coxsackievirus, hepatitis A virus, poliovirus, rhinovirus), polyomaviridae (e.g. BK virus, JC virus), poxviridae (e.g. smallpox), reoviridae (e.g. rotavirus, orbivirus, coltivirus, Banna virus), retroviridae (e.g. HIV), rhabdoviridae (e.g. rabies), togaviridae (e.g. rubella virus), and other enveloped or non-enveloped viruses (e.g. hepatitis D, metapneumovirus, hantavirus, Nipah virus).

Other exemplary pathogens that may be killed or inactivated by the germicidal light source include, for example Aspergillus species, Candida species (e.g. C. albicans, C. glabrata, C. parapsilosis, C. tropicalis, C. krusei), Crytococcus species (e.g. Cryptococcus neoformans, Cryptococcus gattii), other yeasts (e.g. Malassezia, Rhodotorula, Saccharomyces, Saprochaete, Sporobolomyces, and Trichosporon), Dematiaceous fungi, Endemic dimorphic fungi (e.g. Histoplasma capsulatum, Paracoccidioides brasiliensis/lutzii, Blastomyces dermatitidis/gilchristii, Coccidioides immitis/posadasii, Talaromyces [Penicillium] marneffei), and Emmonsia species), dermatophytes, Hyaline molds (Fusarium, Sarocladium, Paecilomyces, Purpureocillium, Scedosporium, Rasamsonia, and Scopulariopsis species) Mucoraceous molds, Pneumocystis jirovecii, and the like.

The germicidal light source may produce any suitable amount of UV-C light, for any suitable duration to kill/inactivate the desired pathogens. Commercially available UV-producing sources, for example, produce anywhere from 0.001-20 Watts of UV light. For example, the UV-producing source may produce about 0.001 to about 1.0 Watts, about 2 Watts, about 3 Watts, about 4 Watts, about 5 Watts, about 6 Watts, about 7 Watts, about 8 Watts, about 9 Watts, about 10 Watts, about 11 Watts, about 12 Watts, about 13 Watts, about 14 Watts, about 15 Watts, about 16 Watts, about 17 Watts, about 18 Watts, about 19 Watts, or about 20 Watts or more of germicidal UV light (e.g. UV-C light).

The germicidal light source may produce germicidal UV light for any suitable duration. For example, the UV light may be produced (e.g. the germicidal light source is activated or turned “on”) for 5 seconds to 60 minutes or longer. For example, the germicidal light may be produced for about 5 seconds, about 10 seconds, about 20 seconds, about 30 seconds, about 40 seconds, about 50 seconds, about 1 minute, about 5 minutes, about 10 minutes, about 15 minutes, about 20 minutes, about 25 minutes, about 30 minutes, about 35 minutes, about 40 minutes, about 45 minutes, about 50 minutes, about 55 minutes, about 60 minutes, two hours, three hours or more (e.g., overnight).

The germicidal light source may be optimized to allow for maximum efficacy in killing/inactivating the desired pathogens without comprising safety. In some embodiments, use of the germicidal light source kills/inactivates 50% or more of pathogens on/in the apparatus or an object placed within the apparatus. For example, the germicidal light source may kill/inactivate at least 50%, at least 55%, at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, of the pathogens on/in the apparatus or on an object placed within the apparatus.

In some embodiments, the multifunctional UV-sanitizing apparatus is a component of a system described herein (e.g., a system comprising a controller and one or more UV-sanitizing apparatuses and optionally one or more UV-irradiating light sources separate from UV-irradiating light of the UV-sanitizing apparatuses). In some embodiments, the multifunctional apparatus is operably connected to a controller of a system described herein. For example, the multifunctional apparatus may be operably connected to a controller (e.g., comprising a processor, a memory component, software, communication means, or the like). The controller may control activation of the one or more germicidal light sources. For example, the processor may run software stored in a local or remote memory component to control activation of the one or more germicidal light sources (e.g., light sources of the one or more UV-sanitizing apparatuses and optionally one or more UV-irradiating light sources separate from UV-irradiating light of the UV-sanitizing apparatuses). Such embodiments may be particularly useful in settings where the germicidal light source is only activated at certain times (e.g. in movie theaters, where the lights (e.g., present within a cupholder or cupholder including a sleeve attached to a chair) are only turned on after hours or between shows; on airplanes when tray tables are stowed within a sleeve and occupants have vacated the cabin; in a hospital, when patients and hospital personnel have vacated a room; or when a hotel room has been vacated). The controller may control activation of the germicidal light sources by wired or wireless communication technology.

In some embodiments, the germicidal light source is coupled to a timer (e.g., a timer located at the light source or a timer run by a controller). For example, the germicidal light source may be coupled to a timer that measures the duration of exposure of the apparatus to the germicidal UV light. The timer may be operably connected to the germicidal light source to control the duration of activation of the germicidal light source. In some embodiments, the timer may instruct the controller to shut off the germicidal light source after a suitable duration of time has passed (e.g. 1-60 minutes). As described herein, the controller may record in a memory component each event of activation of the light source or timer (e.g., record the date, time, duration of exposure of each germicidal light source).

In some embodiments, the multifunctional UV-sanitizing apparatus further includes an illuminating light source (e.g. different from the germicidal light source). The illuminating light source may be disposed on and/or within the apparatus for illuminating the apparatus. Illuminating the apparatus with a light source disposed on and/or within the apparatus significantly facilitates location and proper use of the apparatus during reduced visibility or total darkness conditions, through the use of indirect lighting in a manner which is inconspicuous and non-distracting. For example, such an illuminating light source may be used in cupholders or shelves, or sleeves in seating arrangements, such as in movie theaters, to facilitate location and proper use of the apparatus without being disruptive (e.g., to an occupant of a seating arrangement).

The multifunctional UV-sanitizing apparatus may be in a private location, such as a home or a vehicle. Alternatively, the multifunctional UV-sanitizing apparatus may be in a public location. For example, the multifunctional UV-sanitizing apparatuses described herein may find use in public locations such as airports, airplanes, hotels, healthcare settings (e.g. emergency rooms, doctor offices, etc.), waiting areas in business locations, shared office space, movie theaters, and the like. In some embodiments, the multifunctional UV-sanitizing apparatus may be incorporated into a piece of furniture (e.g. sofa, loveseat, chair, recliner, etc.). In some embodiments, the UV-sanitizing apparatus may be incorporated into a powered piece of furniture (e.g. a powered motion sofa, loveseat, chair, bed, etc.). For example, the multifunctional UV-sanitizing apparatus may be incorporated into a chair in a public location (e.g. airplane seat, bus seat, train seat, subway seat, airport gate area seat, chair in a public lobby, movie theater chair, etc.). In some embodiments, the UV-sanitizing apparatus may be incorporated into a case good (e.g. a nightstand, a desk, a dresser, a bedframe, box spring, a shelving unit, a wall, counter, tv stand/entertainment center, etc.). For example, the UV-sanitizing apparatus may be incorporated into a case good in a private location, such as a boat, car, home, RV, and the like. As another example, the multifunctional UV-sanitizing apparatus may be incorporated into a case good in a public location (e.g. hotel room, a desk in shared office space, a hospital room, public lobby, etc.).

In some embodiments, a piece of furniture containing the multifunctional UV-sanitizing apparatus (e.g. chair, sofa, bed, recliner, etc.) may further comprise one or more motors for control of a component of the piece of furniture (e.g., motor for power-recline, a power headrest, a massage function, and/or a power footrest). In such embodiments, the multifunctional UV-sanitizing apparatus may include a controller operatively connected to the motor for control thereof, with the controller having a user-controllable input for operating the motor. In some embodiments, a controller may be operatively connected to a massage motor for control thereof. The body of a UV-sanitizing apparatus, according to the disclosure, may include a top surface (e.g., a flange), for example, into which a controller, the charging location, and/or other types of controls or connectors may be located.

For any of the embodiments described herein, use of the multifunctional UV-sanitizing apparatuses may be combined with other sanitization methods. For example, the multifunctional UV-sanitizing apparatuses in hotel rooms, airplanes, hospital rooms, etc. may be used in combination with UV-sanitization of the entire room, airplane cabin, etc., or with other chemical or solution-based cleaning.

A multifunctional UV-sanitizing apparatus or system comprising same may further include one or more sensors. For example, a multifunctional UV-sanitizing apparatus may include a pressure sensor. The pressure sensor may be disposed on or within the apparatus. For example, the pressure sensor may be disposed on the bottom surface of a container (e.g., a cupholder, a shelf, a box, a clamshell, etc.) to detect the pressure of an object placed within the container, such as a glass, phone, remote, and the like. As another example, the pressure sensor may be disposed on the single flat surface (e.g. on the tray, table, desk, etc.) to determine whether an object has been placed on the surface. In some embodiments, the pressure sensor may detect pressure and convert this to a signal to be sent to a controller. The controller may receive input from one or more other components of the apparatus (e.g. an on/off switch, a timer, an infrared sensor, other sensors, etc.) or system to control activation of the germicidal light source and/or to control activation of the wireless charging means.

A multifunctional UV-sanitizing apparatus or system comprising same may further include an infrared sensor (e.g., a proximity sensor). For example, an infrared sensor may be disposed on or within the apparatus to detect an object and/or heat emitted from an object placed within or on the apparatus. The infrared sensor may detect heat and convert this to an electrical signal to be sent to the controller. The controller may receive input from other components of the apparatus (e.g. an on/off switch, a timer, a pressure sensor, other sensors, etc.) or system to control activation of the germicidal light source and/or to control activation of the wireless charging means.

In some embodiments, the germicidal light source may be operably controlled by an on/off switch. The switch may be placed in any suitable location on or within the multifunctional apparatus. In some embodiments, the switch may be placed on the single flat surface of the apparatus (e.g. on the tray, table, etc.). In some embodiments, the switch may be placed on an outer surface of the apparatus. In some embodiments, the switch may be placed within the apparatus (e.g. on an inner surface). In some embodiments, the germicidal light source and/or the controller is configured such that the germicidal light source is only activated when the on/off switch is set to “on”. In some embodiments, the status of the one or more additional sensors (e.g. pressure sensor, infrared sensor) and/or a timer may also determine whether the germicidal light source is activated. Such embodiments may vary depending on the intended usage of the apparatus. For example, in some settings (for example, in the home) a safety feature may be to ensure that the apparatus is only turned on when an object of sufficient size and weight is present within the apparatus. In such settings, the germicidal light source and/or the controller may be configured such that the germicidal light source is only activated when the on/off switch is set to “on” and when the one or more sensors detect the presence of an object (e.g. cup, phone, remote) within the apparatus. For example, the controller may be configured such that the germicidal light source is only activated when the on/off switch is set to “on” and when the pressure sensor detects that an object is placed within the apparatus.

In some embodiments, the sensor may be configured such that an object is only detected when the object is larger than a set weight and/or size. For example, the pressure sensor may be configured such that a signal is only sent to the controller when the object weighs more than 100 g. Such embodiments and would be useful in instances wherein the UV-producing source is only intended to be turned on when the switch is turned to on and wherein an object of a suitable size, such as a glass, cup, remote, or phone, is placed within the apparatus. Accordingly, such embodiments would be useful in instances where a smaller object, such as a coin, pencil, pen, children's toy, etc. is present in the apparatus and no UV-sterilization is intended.

In other embodiments, it may be useful for the germicidal light source to be activated only when the multifunctional apparatus is not in use or when a case good or furniture to which the apparatus is attached or integrated is not in use. For example, in public settings such as a movie theater, airport, airplane, bus, train, hotel rooms, and the like, a safety feature of the disclosure may be to ensure that the one or more germicidal light sources are only activated when objects are not present in the apparatus, when objects or people are not on or using the area containing the apparatus, and/or when people are not in the public setting. In such embodiments, the controller may be configured such that the germicidal light sources are only activated when software run by the processor of the controller activates the light sources, or when the one or more additional sensors (e.g. pressure sensor, infrared sensor) do not detect the presence of an object or heat in the apparatus or in the room or rooms in which the apparatus or apparatuses are located, or, similarly, when one or more sensors or other indicators (e.g., time or date) provide information regarding the absence of people on or using the seating containing the apparatus and/or when people are not in the public setting. For example, in a movie theater wherein the seats contain UV-sanitizing apparatuses as described herein, the germicidal light source may only be activated when people are not present in the theater (e.g. between shows, after-hours, etc.). As another example, in an airplane where the tray tables contain UV-sanitizing apparatuses, the germicidal light source may only be activated when people are not present in the airplane (e.g. between flights, during the time when the airplane is typically cleaned with conventional sanitizing procedures). As yet another example, in hotel rooms, hospital rooms, or shared office space, wherein one or more pieces of furniture or case goods (e.g. night-stands, dressers, entertainment centers, desks, seats, beds, etc.) residing in the hotel rooms, hospital rooms, or shared office space contain a UV-sanitizing apparatus and wherein the hotel rooms, hospital rooms, or shared office space optionally contain one or more UV-sanitizing light sources separate from the UV-sanitizing apparatus, the germicidal light sources may be activated only when the room is not occupied (e.g. in between guest stays, patient stays, or during non-use hours of a shared office space).

Thus, in some embodiments, a system disclosed herein (e.g., comprising a controller and one or more germicidal light sources) is configured to control devices or components in addition to and/or in concert with the multifunctional apparatuses. For example, in addition to controlling germicidal light sources present on or within one or more UV-sanitizing apparatuses, a controller of the system may be configured to control one or more germicidal light sources separate from the germicidal light sources on or within the one or more UV-sanitizing apparatuses disclosed herein. For example, in a hotel or dormitory setting, on a plane, or in a shared office space, a germicidal light source located on a ceiling, a wall, a shelf, a counter, an office cubical or partition, a desk, or a floor may be operably connected (e.g., wired or wirelessly connected) to a controller of a system provided herein, where the system activates the germicidal light source located on a ceiling, a wall, a shelf, a counter, an office cubical or partition, a desk, and/or a floor simultaneously with the one or more germicidal light sources on or in the multifunctional apparatuses (e.g., to sanitize not only the multifunctional apparatus and any device or object residing therein, but also to sanitize the room (or cabin in case of a plane), walls, desk, and/or flooring, in the case of shared office space, etc.) in which the multifunctional apparatuses resides.

In some embodiments, a controller (e.g., of a system described herein) directs activation and/or deactivation of the germicidal light source or sources. Such embodiments may be useful in settings wherein multiple UV-sanitizing apparatuses are present, such as in movie theaters, airports, airplanes, buses, trains, hotels, hospitals, shared office space, meeting halls or centers, or other public areas. For example, a controller may direct activation of the germicidal light sources so that the germicidal light source of each UV-sanitizing apparatus (e.g., in a plurality of chairs in a theater or a plurality of desks or nightstands in a hotel or a plurality of tray table storage devices (e.g., a sleeve or pocket apparatus described herein)), and/or the germicidal light source or sources separate from the UV-sanitizing apparatus light source, are each turned on at the same time and turned off at the same time. In a movie theater setting, for example, a controller is can be configured to only turn on the germicidal light source when individuals (e.g., patrons) are not in proximity to the UV-sanitizing apparatus (such as between shows, before the movie theater opens, after the movie theater closes, etc.). As another example, in an airplane a controller can be configured (e.g., it can be programmed) to only turn on the germicidal light source when individuals (e.g., passengers) are not in proximity to the UV-sanitizing apparatus, such as in between flights. As another example, in hotels or hospitals or shared workspace, a controller can be configured to activate each UV-sanitizing apparatus in one or a plurality of rooms or on an unoccupied desk or in an unoccupied bathroom (e.g., on the counter) at the same or different times to sanitize the apparatuses in multiple rooms or multiple desks or multiple surfaces (e.g. counters) (e.g., at the same or different times)).

Some forms of the disclosure include a master multifunctional UV-sanitizing apparatus and one or more slave multifunctional UV-sanitizing apparatuses operatively connected to the master apparatus in such a manner that the germicidal light sources in the slave apparatuses produce germicidal light only when commanded to do so by the master apparatus (e.g., when a switch is turned on to activate the germicidal light or if a sensor is configured to turn the germicidal light on). For example, the master multifunctional apparatus may include one or more sensors (e.g. pressure sensor, infrared sensor) mounted on the body of the master apparatus, and operatively connected to the germicidal light sources of both the master and slave multifunctional apparatuses, for selectively controlling production of germicidal light in both the master and slave apparatuses. For example, the master multifunctional apparatus may control production of germicidal light in both the master and slave apparatuses in such a manner that the sensor causes the germicidal light sources to not produce light when the sensor detects pressure and/or heat in the apparatus. Accordingly, this would prevent unwanted activation of the germicidal light sources in settings, such as a movie theater or an airplane, when one or more users are present. In some embodiments, the master and one or more slave apparatuses are operatively connected to a controller.

In some embodiments, the multifunctional UV-sanitizing apparatus comprises one or more device charging means. For example, the device charging means may be a device charging port or a wireless device charging station. In some embodiments, the multifunctional UV-sanitizing apparatus may comprise one or more ports to facilitate wired device charging. For example, the multifunctional UV-sanitizing apparatus may comprise a USB port (e.g. for use with any suitable USB connector type, including USB-A, USB-B, mini-USB, micro-USB, and/or USB-C). In some embodiments, the multifunctional UV-sanitizing apparatus may comprise one or more wireless charging stations. The device charging means (e.g. device charging port or wireless charging station) maybe placed at any suitable location on or within the apparatus. Representative locations of the device charging means are shown in the accompanying drawings. For example, the device charging means may be placed on an outer surface of the apparatus. In some embodiments, the device charging means may be within the apparatus. For example, the device charging means may be a wireless charging station located within the apparatus, such that the device may be placed within the apparatus for simultaneous charging and sanitization. As another example, the device may be a charging port placed on or within the apparatus. In some embodiments, the device may be plugged into the charging port and placed within the apparatus for simultaneous UV-sanitization and charging. For example, such multifunctional UV-sanitizing apparatuses may simultaneously charge and sanitize phones, remotes, computers, tablets, remotes, and the like.

Wired or wireless charging devices may be operatively connected to a controller. For example, in some embodiments, a wired or wireless charging port placed on or within a multifunctional UV-sanitizing apparatus may comprise means for communicating (e.g., via wired or wireless communication) with the controller. The disclosure is not limited to any particular means for communicating. In some embodiments, the controller is configured to detect and record data from the charging means. For example, a controller can be configured to detect and record each time a device is connected to the charging means (e.g., every time a device is charged using the charging means, duration of charge, identity of the device being charged, information regarding ownership of the device being charged, etc.). A controller can be configured to receive and record (e.g., in a memory component) data or information via a USB port (e.g., used as a charging means). Similarly, a controller can be configured to receive and record data or information via wireless transmissions (e.g., from a phone or tablet). The controller's ability to receive and record data and information from a charging device of one or more multifunctional UV-sanitizing apparatuses permits a system of the disclosure to capture, record, and provide analytics regarding each user of a multifunctional UV-sanitizing apparatus disclosed herein (e.g., in settings where a plurality of UV-sanitizing apparatuses are operated (e.g., shared workspace, airplanes, subways, hospitals, dormitories, hotels and motels, etc.).

In one non-limiting example, a shared workspace comprising a system disclosed herein comprises a plurality of desks with each desk containing a multifunctional UV-sanitizing apparatus described herein (e.g., a multifunctional UV-sanitizing shelf, sleeve, pocket or the like). In some embodiments, each multifunctional UV-sanitizing apparatus is operably connected to a controller (e.g., the charging device of each apparatus is connected to a controller). The controller detects and records data/information from each device connected to (e.g., wired or wirelessly) the apparatus. In this way, the system is used to detect and record each person's device and in turn each person using a desk (e.g., to which the apparatus is connected) of the shared workspace (e.g., location worked, how long worked, etc.). The system can be configured to detect and record use of the UV sanitizing component of the multifunctional UV-sanitizing apparatus (e.g., if the UV sanitizing light is used before, during or after use of a desk). The system may also comprise one or more UV sanitizing light sources separate from the multifunctional UV-sanitizing apparatuses (e.g., one or more UV sanitizing light sources on the ceiling, floor, wall, partitions between desks, etc.). In this way, a system disclosed herein can be used to track the overall use of the shared workspace, the extent to which the multifunctional UV-sanitizing apparatus is being used (e.g., to charge or to sanitize a device on or within a multifunctional UV-sanitizing apparatus), and if and when the shared workspace has been sanitized (e.g., using one or more UV sanitizing light sources separate from the multifunctional UV-sanitizing apparatuses). Optionally, as described herein, the system may comprise one or more alert means (e.g., lights or sounds) for alerting whether or not UV sanitizing light has been used. For example, in some embodiments, if an individual uses a desk of a system described herein comprising multifunctional UV-sanitizing apparatus, and connects to the apparatus (e.g. via wired or wireless connection), the controller detects and records the use. If the individual stops using the desk and fails to activate UV sanitizing light of the UV-sanitizing apparatus and/or a separate UV light source (e.g., on a partition, desk surface, or floor), the controller communicates with the UV sanitizing light source and/or sources and activates an alert (e.g., a red light) on the UV light source and/or sources. If the individual stops using the desk and activates UV sanitizing light of the sanitizing apparatus and/or a separate UV light source (e.g., on a partition, desk surface, or floor), the controller communicates with the UV sanitizing light source and/or sources and activates an alert (e.g., a green light) on the UV light source and/or sources. In this way, the system alerts a subsequent user of the desk whether or not the desk and surfaces associated therewith have been sanitized.

A shared workspace is only one example of an environment in which a system of the disclosure finds use. As described herein, a system of the disclosure can be used in any setting in which a plurality of multifunctional UV sanitizing apparatuses are deployed (e.g., airplanes, subways, hospitals, dormitories, hotels and motels, etc.). A system disclosed herein can be added to (e.g., retrofitted into) any existing environment (e.g., airplane, subway car, hospital room and/or case good located therein, dormitory room and/or case good located therein, hotel or motel room and/or any case good located therein). In this way, existing environments can be made to be safer for users thereof (e.g., by alerting a user that one or more surfaces have or have not been sanitized). In some embodiments, a system of the disclosure is utilized to customize design of a public space (e.g., used to design an airplane, subway car, hospital room and/or case good located therein, dormitory room and/or case good located therein, hotel or motel room and/or any case good located therein) in order to provide users of the public space an environment in which the user is provided real-time knowledge of whether or not a surface upon which a user may have contact has or has not been sanitized.

The present disclosure is not limited to any particular controller. Indeed, a variety of controllers may be utilized to receive (e.g., wired or wireless communication from a sensor, a charging means, a UV light source, etc.), process, and/or send information regarding system usage. For example, controllers that find use in the present disclosure include, but are not limited to, a 32 bit microcontroller (e.g., that utilizes a reduced instruction set computing (RISC) microprocessor). In some embodiments, a controller utilized in the present disclosure integrates a 12-bit analog-to-digital converter (ADC), queued serial peripheral interfaces (QSPI), and/or a four-channel general purpose timer (GPT) (e.g. capable of pulse width modulation (PWM)). The present disclosure is not limited to any particular controller. Indeed, any controller comprising one or more of the functions described herein can be utilized herein.

In some embodiments, a controller stores data in non-volatile flash memory, which communicates with the controller via a serial peripheral interface (SPI) bus. In some embodiments, a controller stores data in a remote location (e.g., in the cloud). During operation of the system, the controller is configured to save and access a variety of data including, but not limited to, UV light activation and/or usage (e.g., UV light of a multifunctional UV-sanitizing apparatus and/or UV light not present on or in a multifunctional UV-sanitizing apparatus), data or information from a device connected to a multifunctional UV-sanitizing apparatus, data or information regarding use of a multifunctional UV-sanitizing apparatus, and/or calibration information. The controller may also be configured to log events into a memory component (e.g., flash memory). In some embodiments, the events logged include device identification information, event, date and time, UV light activation/exposure time, and/or service code.

In some embodiments, a controller is configured to consider one or a plurality of scenarios. For example, a controller can be configured to sort through a look-up table to determine the length of UV-light exposure for a given circumstance (e.g., number of individual UV-light sources to activate based on time of day). In this scenario, system can provide an alert to each UV-light source to indicate that surfaces exposed to light from the UV-light source have been sanitized. In some embodiments, one or more specific conditions (e.g., as monitored and/or recorded by a controller) disable and/or terminate activation of UV-light source of a system disclosed herein. For example, detection of motion by a motion sensor of a system disclosed herein can be used to shut-off one or more UV light sources (e.g., in a shared workspace in which a system disclosed herein has activated all UV light sources in the shared space after-hours, the entry of a person into the workspace detected by a motion sensor is communicated to the controller and the controller terminates activation of one or more UV light sources).

Thus, in some embodiments, the present disclosure provides a system capable of monitoring, recording, and making accessible (e.g., to a user, administrator or other person (e.g., via software that tracks and/or manages data collected, recorded and stored (e.g., by a system))) certain types of data and/or information (e.g., activation and duration of UV sanitizing light use, sanitization status, user information, etc.). For example, a system of the present disclosure provides the ability to monitor information for one or a plurality of multifunctional UV-sanitizing apparatuses and/or one or a plurality of rooms comprising same (e.g., in a time stamped manner. For example, the present disclosure provides the ability to monitor and record the exact timing of when one or more UV-sanitizing apparatuses and/or one or a plurality of rooms comprising same are in use or not in use as well as the identity of a device and/or individual that is using the apparatus or room. The present disclosure also provides the ability to monitor and record whether an individual using an apparatus or system of the disclosure was exposed sanitized or non-sanitized conditions (e.g., using data acquired by a controller of the present disclosure). The present disclosure provides the ability to determine how long an individual was exposes to sanitized or non-sanitized conditions. In some embodiments, software is utilized to database these and other types of information (e.g., in order to create data groupings (e.g., according to user defined fields (e.g., that are used as numerical performance values (e.g., that can be monitored and/or analyzed by a user, administrator or other type of person)))). For example, in some embodiments, information monitored, recorded and/or analyzed by a system described herein can be used (e.g., in data analysis and/or statistical models) to assist users of a system described herein to operate in sanitary conditions. In some embodiments, information and/or data monitored and/or recorded using a system of the present disclosure is utilized by a database organization (e.g., to identify values, trends, averages, etc. (e.g., trends such as not using UV sanitizing light sources, time in a public environment, etc.)). The present disclosure also provides the ability to monitor and record information on a per user basis (e.g., based upon correlation of data collected by a system and the identification of the user(s) (e.g., via user specific information associated with a device in communication with the multifunction UV-sanitizing apparatus)). The disclosure therefore provides, in some embodiments, a system for non-biased human resource (e.g., employee) performance.

In some embodiments, the present disclosure provides software that tracks and/or manages data collected, recorded and stored by a system of the present disclosure. In some embodiments, the software comprises setup, import, search, report and/or backup functionalities. In some embodiments, the software comprises a set-up function that allows a user to configure the program to behave the way the user desires (e.g., collection of data in a specific way (e.g., by date, user, location, multifunctional UV-sanitizing apparatus, etc.). In some embodiments, retrieval of information from a memory component of a system of the present disclosure is password protected. In some embodiments, data can be exported into any type of database (e.g., MICROSOFT EXCEL, ACCESS, SQL database, etc.). In some embodiments, the software comprises import functionalities that permit a user to remove data from the system (e.g., from a memory component of the system (e.g., via USB, cable, wireless technology). In some embodiments, importing data comprises importing information associated with each of the one or more multifunctional UV-sanitizing apparatuses (e.g., that are identified by a serial number assigned (e.g., by the controller) to each). In some embodiments, the software comprises a search function that allows a user to search for specific data (e.g., imported from the memory component). For example, a user can search for data specific to a particular multifunctional UV-sanitizing apparatus, data related to specific events, data related to a specific date and/or time, data related to a specific individual, etc. Thus, the search function allows a user to select only that data that the user is interested in. The software is also configured to permit generation of results based upon search criteria (e.g., tables and/or diagrams for reports).

In some embodiments, software configured to track and/or manage information and/or data collected, recorded and/or stored by a system of the present disclosure is housed and/or run on a personal digital assistant (PDA), a personal computer (PC), a Tablet PC, or smartphone. In some embodiments, the software is configured to run independently of other software. In some embodiments, the software is configured to run within or together with other software including, but not limited to, WINDOWS (e.g., WINDOWS XP, WINDOWS CE, or other WINDOWS based operating system), JAVA, cell phone operating systems, or other type of software. In some embodiments, information and/or data collected, recorded and/or stored by a system of the present disclosure is communicated to a software configured to track and/or manage such information via BLUETOOTH, ZIGBEE, infrared, FM, AM, cellular, WIMAX, WIFI, or other type of wireless technology. In some embodiments, information and/or data collected, recorded and/or stored by a system of the present disclosure is made available over a network (e.g., TCP/IP, SANS, ZIGBEE, wireless, wired, USB, and/or other type of network) or via mobile information recording devices (e.g., flash card, memory stick, disc, jump drive, etc.). In some embodiments, a network is configured to comply with certain government protocols (e.g., Health Insurance Portability and Accountability Act rules and/or regulations, Joint Commission on the Accreditation of Healthcare Organizations rules and/or regulations, Federal Aviation Administration rules and/or regulations, and/or other types of rules and/or regulations). In some embodiments, software configured to interact with a system of the present disclosure comprises a mobile resource for an individual user (e.g., of a shared office space).

Other aspects, objects and advantages of the disclosure will be apparent from the following detailed description and drawings of exemplary embodiments.

### EXPERIMENTAL

The following section provides exemplary embodiments of the disclosure, and should not be considered to be limiting of its scope with regard to alternative embodiments that are not explicitly described herein.

### Example 1

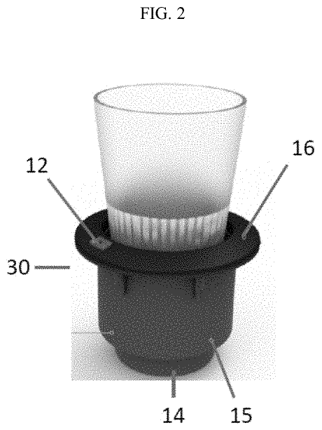

An exemplary multifunctional cupholder apparatus containing multiple germicidal light sources was constructed. FIG. 1 shows a top view image of the exemplary cupholder 30. The cupholder 30 may contain one or more germicidal light sources 10 and/or one or more non-germicidal light sources 11 (e.g., to illuminate the cupholder). The cupholder 30 may further comprise an on/off switch 12 (e.g., that activates timer to control the one or more germicidal lights and/or that turns the one or more germicidal lights 10 on and off. The cupholder 10 may also comprise a controller and/or wiring/circuitry 13. The controller may comprise a microprocessor. The wiring/circuitry may be connected to a separate controller (not shown). FIG. 2 shows a side view image of an exemplary cupholder 30. As shown, the cupholder 30 may contain an on/off switch 12 that controls activation of the germicidal lights. The cupholder 30 comprises a bottom portion 14 and a cupholder body 15 and may also contain space 16 for additional controls and/or charging ports (e.g., a USB port).

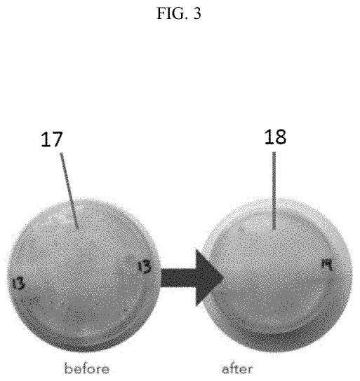

The bacterial-killing capability of the cupholder was evaluated (FIG. 3). The cupholder was swabbed before and after UV-sanitization and bacteria were grown on LB agar plates. The image on the left 17 shows the growth of bacterial colonies swabbed from the cupholder before UV sanitization. The image on the right 18 shows growth of bacterial colonies swabbed from the cupholder after UV sanitization. Following sanitization, 99% of the bacteria were killed.

### Example 2

### Multifunctional UV-Sanitizing Cupholder Apparatus Containing Charging Sleeve

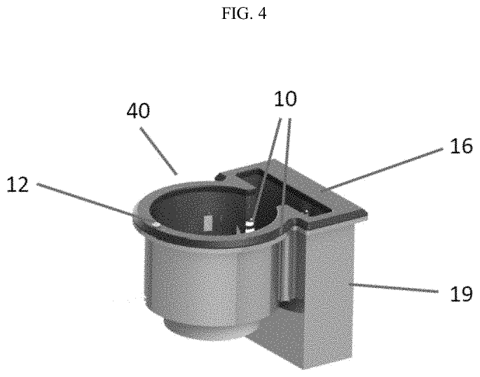

In some embodiments, provided herein are multifunctional UV-sanitizing cupholders with device charging capabilities. For example, a multifunctional UV-sanitizing cupholder may contain a germicidal light source and a wireless charging station and/or a USB port for wired device charging. The wireless charging station or USB port may be placed in any suitable location on the UV-sanitizing cup. For example, as shown in FIG. 4 the cupholder 40 may contain a device charging sleeve 16 that may be fitted to accommodate a mobile phone, a remote control, and the like. The sleeve 16 may comprise a wireless charging station 19 to allow for wired or wireless device charging. Alternatively, the cupholder 40 may contain a USB port on a top surface of the cupholder (e.g., on the top surface of the sleeve 19) and the device may be plugged into the USB port and subsequently stored in the sleeve 19. The multifunctional UV-sanitizing cupholder apparatus 40 may further contain an on/off switch 12 to control the germicidal lights 10, located in any suitable placement on or in the cupholder.

The multifunctional UV-sanitizing cupholder apparatus may be incorporated into a piece of furniture (e.g. sofa, loveseat, chair), a piece of powered motion furniture (e.g., a piece of upholstered power motion furniture such as a power motion sofa, loveseat or chair), or a case good. The cupholder may be placed in a public location (e.g. movie theater seat) or in a private location (e.g. car seat or console, truck, boat, RV, etc.).

### Example 3

### Multifunctional UV-Sanitizing Shelf Apparatus with Charging Capability

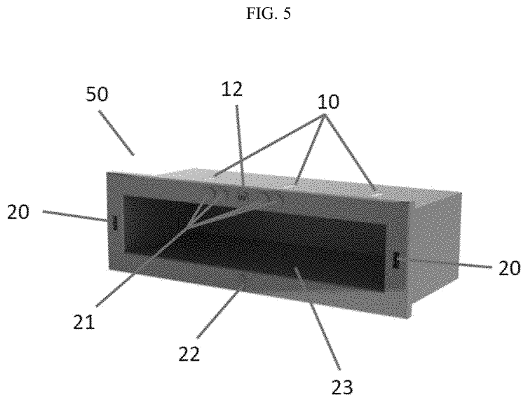

In some embodiments, provided herein are multifunctional UV-sanitizing shelve apparatuses with device charging capabilities. For example, a multifunctional UV-sanitizing shelf apparatus may contain a germicidal light source and a wireless charging station and/or USB port for wired device charging. The wireless charging station and/or USB port may be placed in any suitable location on the shelf. An exemplary shelf apparatus 50 is shown in FIG. 5. The USB port 20 may be placed on an outer surface of the shelf 50. A device may be plugged into the USB port 20 and optionally placed within the shelf 50 for simultaneous charging and sanitization. Alternatively, the inner surface of the shelf 23 may comprise a wireless charging station, such that the device may be placed within the shelf 50 for simultaneous charging and sanitization. The shelf 50 may further comprise an on/off switch 12 for control of the germicidal lights and/or one or more control switches 21 (e.g., for control of a device separate from the shelf apparatus 50). The shelf may comprise an indicator light 22 (e.g., that alerts a user to whether or not the one or more UV sanitizing lights have been activated and/or whether the wireless charging unit is in use).

The shelf may be incorporated into a piece of upholstered furniture (e.g. sofa, loveseat chair, powered motion furniture) or into a case good (e.g. a nightstand, a desk, a dresser, a bedframe, box spring, a shelving unit, a wall, counter, tv stand/entertainment center, etc.). The shelf may be used in a private setting (e.g. car seat or console, truck, boat, RV, etc.) or in a public setting. For example, the shelf may be incorporated into a case good in a public setting, such as a hotel room or a hospital room. The shelf would thereby allow for placement of an object, such as a cell phone or a tv remote, within the shelf and subsequent sanitization of the object by activation of the germicidal lights. In some embodiments, the device may be simultaneously charged and sanitized.

The shelf may be incorporated into any suitable location within the piece of furniture and/or case good. Alternatively, the shelf may be placed on top of, or affixed under the piece of furniture and/or case good.

The shelf may be any suitable size. For example, the shelf may be a suitable size for placement and charging of a mobile phone, a remote control, a laptop, a tablet, etc.

### Example 4

### Multifunctional UV-Sanitizing Box/Clamshell Apparatus with Charging Capability

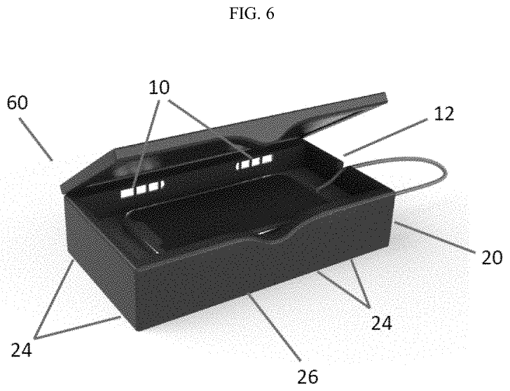

In some embodiments, provided herein are multifunctional UV-sanitizing boxes/clamshell apparatuses with device charging capabilities. For example, a multifunctional UV-sanitizing box/clamshell apparatus contains a germicidal light source and a wireless charging station and/or USB port for wired device charging. The wireless charging station and/or USB port may be placed in any suitable location. For example, as shown in FIG. 6 the box 60 may comprise a USB port located on an outer surface of the box 20. A device (e.g. mobile phone, remote, etc.) may be plugged into the USB port and subsequently placed within the box 60 for simultaneous charging and sanitization. Alternatively, the box/clamshell 60 may contain a wireless charging station 26 inside of the box/clamshell, such that the device may be placed on top of the wireless charging station for charging. The lid of the box/clamshell may be closed prior to activation of the germicidal lights 10 to ensure user safety while the lights are on. The multifunctional box/clamshell apparatus 60 may further contain an on/off switch 12 to control activation of the germicidal lights. One or more screw holes 24 may be present on the box 60 (e.g., for mounting on or in a case good).

The multifunctional box/clamshell apparatus may be incorporated into the center console of a car, boat, RV, sofa, etc. The multifunctional box/clamshell apparatus may be incorporated into a case good or attached to a case good. The box/clamshell may be used in private settings (e.g. car seat or console, truck, boat, RV, etc.) or in a public setting (e.g. center console on bus seats, train seats, etc.).

The box/clamshell may be any suitable size. For example, the shelf may be a suitable size for placement and charging of a mobile phone, a remote control, a laptop, a tablet, etc.

### Example 5

### Multifunctional UV-Sanitizing Pocket Apparatus with Charging Capability

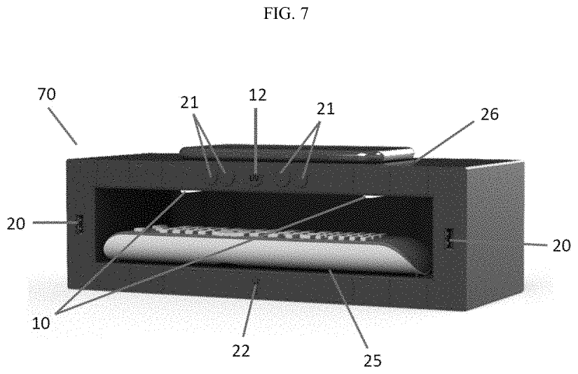

In some embodiments, provided herein are multifunctional UV-sanitizing pocket apparatuses with device charging capabilities. For example, a multifunctional UV-sanitizing pocket apparatus may contain a germicidal light source and a wireless charging station and/or USB port for wired device charging. The wireless charging station and/or USB port may be placed in any suitable location on the pocket. For example, as shown in FIG. 7 the USB port 20 may be placed on an outer surface of the pocket 70. The device may be plugged into the USB port 20 and optionally placed within the pocket 25 for simultaneous charging and sanitization. Alternatively, the inner surface of the pocket 25 (e.g. the location on which a cell phone or remote is resting in FIG. 7) or the exterior surface of the pocket 26 may comprise a wireless charging station, such that the device may be placed within the pocket 25 or on top of the pocket 26. In some embodiments, the pocket may be used for simultaneous charging and sanitization of an object. The pocket may further comprise an on/off switch 12 for control of the germicidal lights 10, and/or one or more control switches 21 (e.g., for control of a device separate from the shelf apparatus 50). The shelf may comprise an indicator light 22 (e.g., that alerts a user to whether or not the one or more UV sanitizing lights have been activated and/or whether the wireless charging unit is in use).

The multifunctional pocket apparatus may be incorporated into a piece of furniture (e.g. sofa, loveseat chair, powered motion furniture) or into a case good (e.g. a nightstand, a desk, a dresser, a bedframe, box spring, a shelving unit, a wall, counter, tv stand/entertainment center, etc.). The multifunctional pocket apparatus may be used (e.g. in a car seat or console, truck, boat, RV, etc.) in a private setting or in a public setting. For example, the multifunctional pocket apparatus may be incorporated into a case good in a public setting, such as a hotel room or a hospital room. The multifunctional pocket apparatus allows for placement of an object, such as a cell phone or a tv remote, within the pocket and subsequent sanitization of the object by activation of the germicidal lights. In some embodiments, the device may be simultaneously charged and sanitized.

### Example 6

### Multifunctional UV-Sanitizing Sleeve Apparatus with Charging Capability

### Example 6

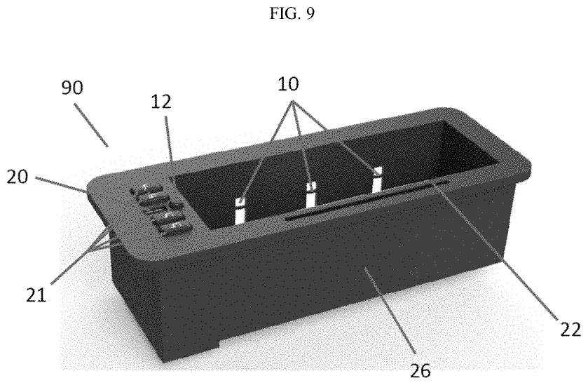

In some embodiments, provided herein are multifunctional UV-sanitizing sleeve apparatuses with device charging capabilities (See, e.g., FIGS. 8 and 9). For example, a multifunctional UV-sanitizing sleeve apparatus 80, 90 may contain a germicidal light source 10 and a wireless charging station 26 and/or USB port 20 for wired device charging. The wireless charging station and/or USB port may be placed in any suitable location in the sleeve. For example, as shown in FIG. 9, the USB port 20 may be placed on an outer surface of the sleeve 90. The device may be plugged into the USB port 20 and optionally placed within the sleeve for simultaneous charging and sanitization via UV light 10 irradiation. In some embodiments, the sleeve is used for simultaneous charging and sanitization of an object. The sleeve may further comprise an on/off switch 12 for control of the germicidal lights, and/or one or more control switches 21 (e.g., for control of a device separate from the sleeve apparatus 80, 90). The sleeve may comprise an indicator light 22 (e.g., that alerts a user to whether or not the one or more UV sanitizing lights have been activated and/or whether the wireless charging unit is in use).

The multifunctional sleeve apparatus may be incorporated into a piece of furniture (e.g. sofa, loveseat chair, powered motion furniture) or into a case good (e.g. a nightstand, a desk, a dresser, a bedframe, box spring, a shelving unit, a wall, counter, tv stand/entertainment center, etc.). The multifunctional sleeve apparatus may be used (e.g. in a car seat or console, truck, boat, RV, etc.) in a private setting or in a public setting (e.g., incorporated into seating of a plane, subway, bus, etc.). For example, the multifunctional sleeve apparatus may be incorporated into a case good in a public setting, such as a hotel room or a hospital room. The multifunctional sleeve apparatus allows for placement of an object, such as a cell phone or a tv remote, within the sleeve and subsequent sanitization of the object by activation of the germicidal lights. In some embodiments, the device may be simultaneously charged and sanitized.

One of skill in the art understands well that the components described below can be altered, for general operation or for specific situational purpose. Combinations of the following embodiments, as well as additional features, are within the scope of the disclosure. The following examples should be viewed as exemplary and not limiting.

Although the disclosure has been described in connection with specific preferred embodiments, it should be understood that the claims should not be unduly limited to such specific embodiments. Indeed, various modifications of the described modes for carrying out aspects of the disclosure that are obvious to those skilled in the relevant fields are intended to be within the scope of the following claims.

What is claimed is:

1. A case good or upholstered seating arrangement comprising a multifunctional UV-sanitizing apparatus, wherein the multifunctional UV-sanitizing apparatus contains:
a sleeve, a pocket or a shelf;
a device charging means; and
at least one germicidal light source;
wherein the germicidal light source is disposed on the multifunctional UV-sanitizing apparatus in order that it irradiates the sleeve, pocket or shelf with UV-sanitizing light when activated.

2. The case good or upholstered seating arrangement of claim 1, wherein the multifunctional UV-sanitizing apparatus comprises two or more germicidal light sources.

3. The case good or upholstered seating arrangement of claim 1, wherein the germicidal light source produces 0.001-20 Watts of UV-C light.

4. The case good or upholstered seating arrangement of claim 1, wherein the device charging means of the multifunctional UV-sanitizing apparatus comprises a device charging port.

5. The case good or upholstered seating arrangement of claim 4, wherein the device charging port is a USB port.

6. The case good or upholstered seating arrangement of claim 4, wherein the device charging means of the multifunctional UV-sanitizing apparatus comprises a wireless charging station.

7. The case good or upholstered seating arrangement of claim 1, wherein the multifunctional UV-sanitizing apparatus further comprises a timer.

8. The case good or upholstered seating arrangement of claim 1, wherein the upholstered seating arrangement is a powered sofa, loveseat or chair.

9. The case good or upholstered seating arrangement of claim 1, wherein the case good is selected from the group consisting of a nightstand, a desk, a dresser, a bedframe, a box spring, a head board, a shelving unit, a wall, a counter, a tv stand or entertainment center, a chair, and a seat.

10. The case good or upholstered seating arrangement of claim 1, wherein the case good is a desk located in a shared office space, wherein the shared office space comprises:
a plurality of desks, with each desk of the plurality of desks comprising a multifunctional UV-sanitizing apparatus;
one or more shared office space irradiating-germicidal light sources separate from the germicidal light source of the multifunctional UV-sanitizing apparatus;
one or more partitions separating the plurality of desks; and
a controller operably connected to:
each multifunctional UV-sanitizing apparatus of the plurality of desks; and
the one or more shared office space irradiating-germicidal light sources.

11. The case good or upholstered seating arrangement of claim 10, wherein the desk and/or one or more partitions comprise a germicidal light source for irradiating the desk surface that is separate from both the germicidal light source of the multifunctional UV-sanitizing apparatus and the one or more shared office space irradiating-germicidal light sources.

12. The case good or upholstered seating arrangement of claim 1, further comprising at least one control for controlling a component of the case good or upholstered seating arrangement separate from the multifunctional UV-sanitizing apparatus.

13. A system comprising:
A) a plurality of case goods and/or upholstered seating arrangements, each case good or upholstered seating arrangement comprising a multifunctional UV-sanitizing apparatus comprising:
a device charging means; and
at least one germicidal light source; and
B) a controller comprising a processor, a communication means for sending and receiving data and/or information, and a memory component.

14. The system of claim 13, wherein the plurality of case goods are selected from the group consisting of a nightstand, a desk, a dresser, a bedframe, a box spring, a head board, a shelving unit, a wall, a counter, a tv stand or entertainment center, a chair, a seat, and combinations thereof.

15. The system of claim 13, wherein one or more of the plurality of case goods are located in a plurality of rooms, wherein the controller is configured to control the activation and deactivation of the at least one germicidal light source of each of the multifunctional UV-sanitizing apparatuses.

16. The system of claim 15, wherein the plurality of rooms each contain one or more room germicidal light sources located on a ceiling, a floor, a wall, a counter, and/or on a desk in the room, and wherein the controller is configured to control the activation and deactivation of the at least one germicidal light source of each of the multifunctional UV-sanitizing apparatuses and the room germicidal light sources.

17. The system of claim 13, wherein the controller monitors and records data and/or information regarding the activation and/or deactivation of the at least one germicidal light source of each of the multifunctional UV-sanitizing apparatuses and/or the room germicidal light sources.

18. The system of claim 13, wherein the plurality of case goods are a plurality of desks located in a shared office space, with each desk of the plurality of desks comprising at least one of the multifunctional UV-sanitizing apparatuses; wherein the shared office space comprises one or more shared office space irradiating-germicidal light sources separate from the germicidal light source of the multifunctional UV-sanitizing apparatuses; and wherein the controller receives and records data and/or information regarding the activation and/or deactivation of the at least one germicidal light source of each of the multifunctional UV-sanitizing apparatuses and/or the shared office space irradiating-germicidal light sources.

19. The system of claim 13, further comprising:
C) an alert means for alerting whether or not UV-sanitizing light was activated.

20. The system of claim 13, wherein the plurality of case goods are a plurality of nightstands individually located in separate rooms of a hotel, with each nightstand comprising at least one of the multifunctional UV-sanitizing apparatuses; wherein each room of the hotel comprises one or more room irradiating-germicidal light sources separate from the germicidal light source of the multifunctional UV-sanitizing apparatuses; and wherein the controller receives and records data and/or information regarding the activation and/or deactivation of the at least one germicidal light source of each of the multifunctional UV-sanitizing apparatuses and/or the room irradiating-germicidal light sources.
